(19) United States
(12) Patent Application Publication
(10) Pub. No.: US 2010/0069895 A1
(43) Pub. Date: Mar. 18, 2010

(54) DEVICE AND METHOD FOR TREATING A PART OF A HUMAN OR ANIMAL BODY IMPLEMENTING TREATMENT DOSE DELIVERY MEANS AND DOSIMETRY CONTROL MEANS

(75) Inventors: Jaouad Zemmouri, Genech (FR); Philippe Rochon, Loffre (FR); Liliane Lefevre, Lille (FR); Serge Mordon, Mouvaux (FR); Benjamin Wassmer, Wattiginies (FR)

Correspondence Address: HARNESS, DICKEY & PIERCE, P.L.C., P.O. BOX 828, BLOOMFIELD HILLS, MI, 48303, US

(73) Assignee: OSYRIS MEDICAL, Hellemmes Lille (FR)

(21) Appl. No.: 12/396,990

(22) Filed: Mar. 3, 2009

Related U.S. Application Data
(60) Provisional application No. 61/145,299, filed on Jan. 16, 2009.

(30) Foreign Application Priority Data
Sep. 16, 2008 (EP) 08 370 019.5

(51) Int. Cl. A61B 18/18; A61B 5/05

(52) U.S. Cl. 606/9; 600/409

(57) ABSTRACT

Device for treatment of a part of a human or animal body, including:
treatment dose delivery means enabling treatment doses to be delivered to the human or animal body,
mapping means enabling a zone or a volume of the body to be treated to be mapped and spatially defined in a predefined frame of reference and in a form of mapping data,
localisation means enabling the instantaneous position of the treatment dose delivery means outlet to be localised in the form of localisation data in the frame of reference,
electronic control means, which, are suitable for recording the mapping data of at least one zone or one volume of the body to be treated, acquired with the help of the mapping means, and which, enable a treatment to be controlled by using, during the course of the treatment, at least the mapping data and the localisation data.

### CROSS-REFERENCE TO RELATED APPLICATIONS

This application claims the benefit of U.S. Provisional Application No. 61/145,299, filed on Jan. 16, 2009. This application claims the benefit and priority of European Patent Application No. 08 370 019.5 filed Sep. 16, 2008. The entire disclosure of each of the above applications is incorporated herein by reference.

### FIELD OF THE INVENTION

The present invention relates to the field of treatment of a human or animal body by means of an instrument that enables treatment doses to be delivered locally to a part of the human or animal body. In this field, the invention relates more particularly, but not exclusively, to the field of treatment of a part of the human or animal body via cutaneous, sub-cutaneous or intra-cutaneous irradiation by means of electromagnetic radiation, the treatment doses in this case corresponding to the energies of said electromagnetic radiation delivered to the different positions of the instrument in the zone or the volume treated. Within the context of the invention, and according to the type of instrument used, the treatment can be a therapeutic, prophylactic or cosmetic treatment of the non-invasive type, or an invasive therapeutic, prophylactic or cosmetic treatment, for instance sub-cutaneous or intra-cutaneous, as for instance adipocytolysis, lipolysis, endovenous treatments, skin remodelling or skin healing through heating the collagen present in the dermis, or liposuction.

### PRIOR ART

There are numerous different treatments during which a treatment dose is delivered locally to a part of the human or animal body, for cosmetic, therapeutic or prophylactic purposes of said part of the human or animal body, and by using a specific medical instrument, also commonly referred to as a “hand piece” and adapted in particular to the nature of the treatment doses that are to be delivered.

The treatment dose can for instance be a dose of electromagnetic radiation; in this case, the delivered dose corresponds to the energy of electromagnetic radiation applied to a given position of the instrument in a treatment zone or volume. The treatment dose can for instance be a dose of a substance or chemical product administered locally to said part of the human or animal body; in this case, the delivered dose corresponds to the quantity of the substance or chemical product administered at each position of the instrument.

These different therapeutic, prophylactic or cosmetic treatments can be classed into two different categories, depending on whether they are of the invasive or the non-invasive type.

Among the invasive type treatments, one can cite all the therapeutic, prophylactic or cosmetic treatments based on an intra-cutaneous or sub-cutaneous irradiation of the zone that is to be treated by means of electromagnetic radiation, and in particular based on an irradiation by means of an electromagnetic radiation produced for instance in the visible wavelength region by using a continuous or pulsed laser beam with different power levels. In these intra-cutaneous or sub-cutaneous treatments, electromagnetic radiation is introduced into the dermis or under the dermis to the zone or volume to be treated, by means for instance of a hollow needle or a cannula, in which an optical fibre is inserted and linked to an adapted source of electromagnetic radiation, for instance a laser. Then the treatment is carried out by pulling, in a continuous or discontinuous manner, the cannula/optical fibre or needle/optical fibre unit, and by activating the laser source so as to perform laser shots at different positions of the distal emission extremity of the optical fibre during the continuous or discontinuous movement of withdrawing the cannula/optical fibre or needle/optical fibre unit.

More particularly, treatments by sub-cutaneous electromagnetic radiation can include primarily, but not exhaustively, adipocytolysis and lipolysis, which consist in treating, in particular by the effect of heat, the adipose cells present in the hypodermis, by inserting into the hypodermis, at different depths, the distal extremity of the optical fibre, through which the electromagnetic radiation exits. Lipolysis enables a destruction of the adipocytes by the effect of electromagnetic radiation. This destruction results in a liquefaction of the fat in the zones in proximity to the passage of the optical fibre, where the temperature has been elevated to a sufficiently high level (50-70° C.) to destroy the membranes of the adipocytes and release the triglycerides. The term adipocytolysis is used to describe the medium and long-term effect of elevating temperature on the adipocytes. In effect, in the zones further away from the passage of the optical fibre, the temperature elevation is less high (40-50° C.). Nevertheless, these temperatures induce heat stress in the adipocytes that will trigger in months following the treatment an apoptosis of the adipocytes. This progressive cellular death thus provokes a loss of volume in the treated fatty tissue, which reaches its maximum 6 to 8 months after the intervention. In this particular type of invasive treatment, adipocytolysis is the major effect of electromagnetic radiation and lipolysis makes but a small contribution to the reduction of fatty volume.

Any endovenous therapy, in which electromagnetic radiation is produced in a vein, can also be included.

For laser lipolysis, the following publications can for instance be referred to: U.S. Pat. No. 6,206,873, U.S. Pat. No. 5,954,710, US 2006/0224148. For endovenous laser therapy, publications U.S. Pat. No. 4,564,011, U.S. Pat. No. 5,531,739, U.S. Pat. No. 6,398,777 can for instance be referred to.

Treatments of the invasive type can also include skin tightening treatments obtained by delivering electromagnetic doses under the dermis.

Treatments of the invasive type can also include all therapeutic, prophylactic or cosmetic treatments consisting in locally administering a chemical product or substance to a part of the human or animal body by means of a syringe-like instrument.

Treatments of the non-invasive type include in particular all therapeutic, prophylactic or cosmetic treatments implementing an external irradiation of a part of the human or animal body through electromagnetic radiation, for instance by means of an exolaser. In particular, in the field of dermatology, this applies to all skin heat treatments of the non-invasive type.

For instance, it is known to implement non-invasive heat treatments to heat the collagen present in the dermis of the skin.

A significant application of these non-invasive heat treatments of the dermis is the remodelling of the skin through collagen in order to reduce or get rid of wrinkles due to ageing, or to suppress unsightly aspects of the skin, so-called “orange peel skin.”

U.S. Pat. Nos. 6,659,999 and 7,094,252, for instance, suggest skin remodelling solutions through collagen based on external electromagnetic radiation of the skin by means of an exolaser.

Regardless of the type of treatment (invasive or non-invasive), the therapeutic, prophylactic or cosmetic effect depends on the treatment doses, which are in effect delivered to the part of the human or animal body, but also on the localisation and distribution of these doses. These treatments are thus called dose-dependent. An underdose can render the therapeutic, prophylactic or cosmetic treatment less effective, even ineffective. Conversely, an overdose can result in irreversible damage being caused to the treated zone and for instance trigger an irreversible and detrimental destruction of certain healthy tissues or healthy cells. The overdosing or underdosing does not depend solely on the dose delivered at each position of the instrument, but also depends on the localisation and distribution of these doses. The localisation and distribution of the doses depend on the manner in which the practitioner manipulates the instrument during the course of the treatment. In effect, if the practitioner who performs the treatment commits a localisation mistake and delivers correct treatment dose quantities in different positions, but if all or part of these positions are situated outside the zone or volume to be treated, or if he erroneously forgets to treat a zone or a volume, or else if during the course of the treatment he displaces the treatment dose delivery instrument too quickly or, conversely, not quickly enough, resulting in the incorrect distribution of the delivered doses, the therapeutic, prophylactic or cosmetic treatment can be less effective or ineffective, and even prove to be dangerous in certain cases.

It is thus essential for the success and harmlessness of the treatment to be able effectively to control not only the treatment doses that are actually delivered to different treatment positions, but also to be able to control the localisation and distribution of the different doses in a frame of reference linked to the human or animal body.

More particularly, a major difficulty of local electromagnetic radiation treatments (external or internal) of a part of a human or animal body is linked to the risks of irreversibly destroying, through the effect of heat, non-targeted cells in the treated zone, or even in a zone adjoining the treated zone. This risk is dependent not only on the power and the wavelength of the electromagnetic radiation, but also and primarily on the speed with which the electromagnetic radiation spot is displaced to the zone to be treated. The latter parameter of the speed of displacement, however, most often depends on a human manual action performed by the practitioner carrying out the treatment and is thus a significant source of risk.

Attempts to resolve this difficulty to date include efforts to control the energy of the electromagnetic radiation applied during treatment. In US patent application 2004/0199151, for instance, a solution is proposed based on measuring the speed of withdrawal of the optical fibre and on an automatic control of the laser power, as a function of the measured speed, so as to maintain a suitable constant treatment energy. Different solutions for measuring the displacement speed of the optical fibre are considered. For instance, specific marks made upon a certain length of the optical fibre are automatically detected or an optical speed-measuring device, through which the optical fibre passes, is implemented. This solution has two disadvantages. On the one hand, the measuring means of the displacement speed of the optical fibre are positioned in the field of surgery, which brings about a problem of sterility of these measuring means. On the other hand, this solution does not enable the zone actually treated to be localised in a frame of reference linked to the human or animal body, and does not enable the distribution of the energy doses in the zone actually treated to be known.

Other control solutions based on external detection of the skin temperature by means of an infrared sensor or by thermosensitive reagents applied on the skin have also been suggested. These solutions are not satisfactory, however, due in particular to the time required for the heat to be propagated to the surface of the skin. Once the skin temperature threshold is reached and detected, it is generally too late and irreversible sub-cutaneous thermal lesions may already have been caused.

International patent application WO 2006/107522 suggests a solution for laser lipolysis, in which the laser beam is introduced into the hypodermis by means of a cannula/optical fibre unit. One objective in this publication is to protect the dermis against the destructive thermal effects of the laser beam by ensuring that the distal extremity of the optical fibre, upon firing, is not situated in the dermis, but rather in the hypodermis, at a sufficient distance from the dermis. To this effect, the depth of the laser shot is controlled by detecting, by means of an external optical sensor, the intensity of the light energy of the shot, which passes through the different layers (hypodermis, dermis, epidermis) and which is visible from the outside because of the sensor. The greater the intensity, the more shallow the laser shot. This solution does not, however, allow a localisation of the zone actually treated in a frame of reference linked to the human or animal body and does not enable the distribution of the energy doses in the zone actually treated to be known.

International patent application WO 2007/027962 and International patent application WO 2005/063138 incidentally also suggest a hand piece including an optical fibre which is linked to a laser source, and which enables an external part of a human body to be treated by electromagnetic radiation. This hand piece is in addition equipped with optical detection means enabling the absolute position of the hand piece or variations of certain positioning parameters of the hand piece to be detected, such as variations in position, variations in angle or variations in speed of displacement of the hand piece. This information is used for instance for an automatic control of the power of the laser. The information on the absolute position of the hand piece or the variations of the positioning parameters of the hand piece does not enable the actually treated zone to be localised in a frame of reference linked to the human or animal body, and as a result cannot be used to control that the zone or the volume to be treated has in actual fact been treated with the correct treatment doses.

International patent application WO 2007/027962 also teaches the cartography of the different positions of the hand piece during the course of the treatment. However, this cartography by itself does not enable a control of whether the treatment has been carried out in the correct zone of the human or animal body. This control of the correct localisation of the treatment on the human or animal body must be carried out by the practitioner by visually controlling the position of the hand piece in relation to the human or animal body.

Such a visual control of the positioning of the hand piece is possible in the case of a non-invasive treatment, but is not adapted for a treatment implemented by means of an invasive instrument. In the case of invasive treatments, and in contrast to non-invasive treatments, the practitioner cannot easily visually control the position of his instrument in the treated zone or volume at every instance of time during the course of treatment. It is thus impossible for him to visually control the localisation of the different doses that are being delivered in a reliable way. Up to the present day there is thus a need for a technical solution enabling the movement of the practitioner to be guided during the course of an invasive treatment so that he can ensure that the treatment doses are being delivered to the right place within the zone or volume to be treated.

A solution for guiding the movement of the practitioner during an invasive treatment consists in practising the movement while controlling it by means of a medical imagery system of the MRI type. This type of solution, however, imposes the use of a very costly and voluminous medical imagery device, which limits its use.

### OBJECTIVE OF THE INVENTION

An objective of the invention is to suggest a new technical solution, which facilitates and improves the control of treatment doses delivered to a part of the human or animal body, and which can be implemented with any type of treatment dose delivery means used in a dose-dependent treatment, i.e. with treatment dose delivery means of the invasive type as well as of the non-invasive type.

### SUMMARY OF THE INVENTION

A first object of the invention is thus a device for treatment of a part of the human or animal body as defined in claim 1.

This treatment device comprises:
treatment dose delivery means enabling treatment doses to be delivered to a part of the human or animal body,
mapping means enabling a zone or a volume of the human or animal body to be treated to be mapped and spatially defined in a predefined frame of reference (Rt) and in the form of mapping data (Pᵢ),
localisation means enabling the instantaneous position of the treatment dose delivery means outlet to be localised in the form of localisation data (P′) in the said frame of reference (Rt),
electronic control means, which, on the one hand, are suitable for recording said mapping data (Pᵢ) of at least one zone or one volume of the human or animal body to be treated, acquired with the help of said mapping means, and which, on the other hand, enable a treatment to be controlled by using, during the course of the treatment, at least said mapping data (Pᵢ) and said localisation data (P′).

In the context of the invention, the treatment can consist of treating the part of the human or animal body with electromagnetic waves, electric waves or mechanical waves, and more particularly acoustic waves; in this case the treatment dose is the energy of the treatment wave that is delivered at a given position of treatment dose delivery means. The treatment can also consist in the administration of a substance or a chemical product into said part of the human or animal body; in this case the treatment dose is the quantity of the substance or chemical product administered at each position of the treatment dose delivery means.

The invention also has as a further object a treatment method as defined appended claims.

The invention also has as its object the use of the above-cited device or the above-cited method to carry out a treatment from among the following: sub-cutaneous or intra-cutaneous treatment, endovenous therapy, destroying adipose cells, lipolysis treatment, adipocytolysis treatment, heating of collagen in the dermis, cosmetic skin remodelling or skin healing treatment through heating the collagen present in the dermis.

### BRIEF DESCRIPTION OF DRAWINGS

Other characteristic features of the invention will appear more clearly upon reading the detailed description hereinafter of several embodiments of the invention given by way of non-limiting and non-exhaustive examples, said description being given with reference to the appended figures, in which:

FIG. 1 shows, in a schematic manner, an example of a medical device of the invention enabling an invasive laser treatment of a part of the human body,

FIG. 2 shows an example of a medical instrument including a hand piece enabling the manipulation of a cannula/optical fibre unit,

FIG. 3 is a synoptic example of the main electronic components of the treatment device of FIG. 1,

FIGS. 4 and 5 show algorithms illustrating the main functioning stages of the device of FIG. 1,

FIG. 6 shows an example of a display of the zone to be treated, which has been mapped out, before the delivery of a first treatment dose,

FIG. 7 shows an example of a display of the zone to be treated, which has been mapped out, and of the cartography of the doses that are delivered during the course of a treatment.

### DETAILED DESCRIPTION

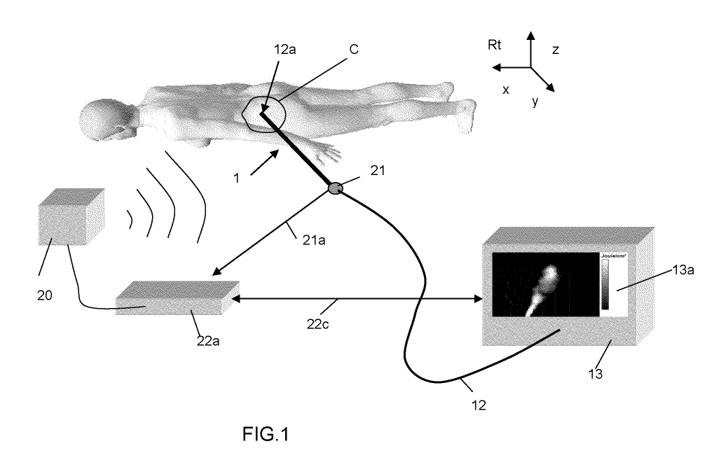

FIG. 1 shows, in a schematic manner, an example of a medical device according to the invention, which enables a part of a human body C to be treated.

In this particular example, but in a non-limiting and non-exhaustive manner of the invention, the treatment device enables different types of invasive laser treatment of the human body to be carried out. These treatments can include, in a non-exhaustive manner, laser adipocytolysis, laser lipolysis, endovenous laser therapies, laser skin remodelling, skin healing through heating the collagen present in the dermis and/or by laser heat stimulation of fibroblasts enabling the speeding up of collagen production in the dermis.

### Treatment Device

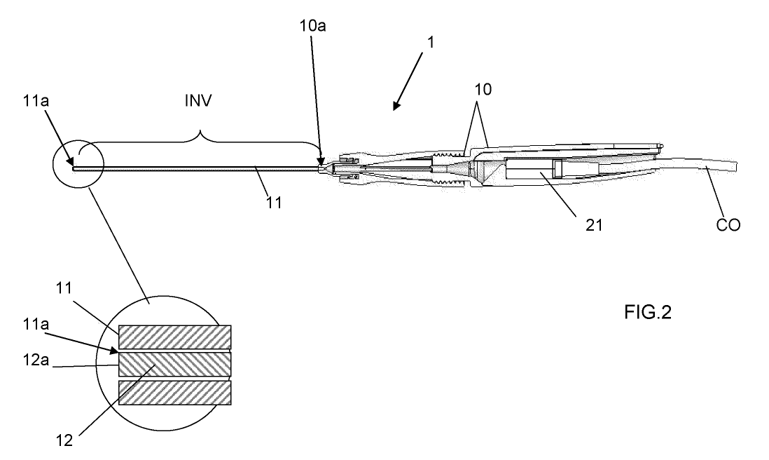

This treatment device comprises treatment dose delivery means including an instrument 1 that can be manipulated by hand and that displays for instance the particular structure of FIG. 2, and an electromagnetic radiation source 13b.

With reference to FIG. 2, the instrument 1 includes for instance a hand piece 10 on which a cannula 11 is fastened, and an optical fibre 12, which is threaded into the hand piece 10 and the cannula 11 and which is immobilised with regard to said cannula. The distal extremity 12a of the optical fibre constitutes the outlet of the instrument 1 through which the electromagnetic radiation doses are delivered. In the particular example illustrated in FIG. 2, the distal extremity 12a of the optical fibre 12 is flush with the distal opening 11a of the cannula. In a further embodiment, the distal extremity 12a of the optical fibre 12 can be situated at the exterior of the cannula 11, but in immediate proximity to the distal opening 11a of the cannula 11.

The hand piece 10 enables the cannula 11/optical fibre 12 unit to be manipulated by hand and constitutes a non-invasive part of the instrument 1. The part of the cannula 11/optical fibre 12 unit, referenced “INV” on FIG. 2, which is external to the hand piece 10 and which extends from the distal extremity 10a of the hand piece 10, constitutes an invasive part of the instrument 1 destined to be partially or totally introduced into the part of the human body C to be treated.

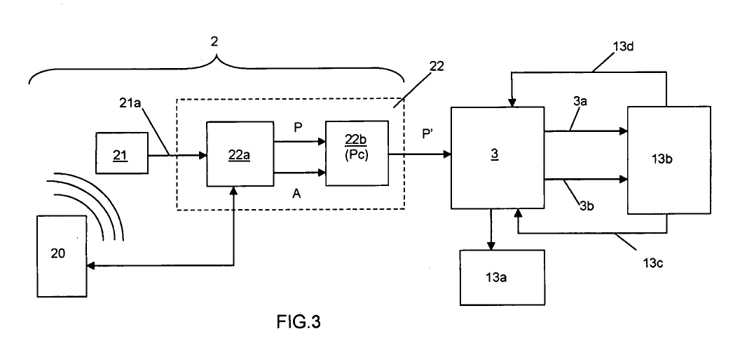

The optical fibre 12 is connected at its other extremity to the electromagnetic radiation source (FIG. 3—laser source 13b), which is integrated with a device 13 that also includes a screen 13a for the visualisation of the laser treatment. The emission frequency of the source 13b will be chosen in a known manner by a person skilled in the art and can, according to the type of treatment, be in the visible, infrared, hyperfrequency or radiofrequency region. The emission frequency and/or power of the electromagnetic radiation source are preferably adjustable. When the electromagnetic radiation source is in operation, the instrument 1 delivers at its distal emission extremity 12a an electromagnetic radiation that can be applied to a part of the body C to be treated.

With reference to FIGS. 1 and 3, the treatment device also includes:
localisation means 2 of the instantaneous 3D position P′(x′(t), y′(t), z′(t)) of the outlet 12a of the optical fibre 12 in a predefined frame of reference (Rt), constituted in this particular embodiment by the three-dimensional reference point (X, Y, Z),
and electronic means 3 that communicate with the localisation means and enable the treatment carried out by means of the instrument 1 to be controlled.

A particular embodiment of the localisation means 2 and the electronic means 3 will be detailed hereinafter.

### Localisation Means 2

With reference to FIGS. 1 and 3, the localisation means 2 include a magnetic field transmitter 20, a sensor 21 fastened to the instrument 1, at a different position to the outlet position 12a of the optical fibre 12, and electronic computation means 22.

More particularly, with reference to the particular example of FIG. 2, the sensor 21 is housed inside the hand piece 10. In this FIG. 2, an electrical cord CO is shown, which is connected to the sensor 21 and which contains on the one hand the electrical leads for supplying the sensor and on the other hand the electrical leads transporting the electrical signals 21 a delivered by the sensor 21.

While operating, the transmitter 20 is fastened and positioned in proximity to the body C and emits a magnetic field, which is received by the sensor 21. The sensor 21 is sensitive to the magnetic field produced by the transmitter 20 and delivers electrical signals 21a, which are characteristic of its absolute instantaneous position and its absolute instantaneous angle of inclination in said magnetic field. These electrical signals 21a are received and treated by the first electronic computation means 22a which are suitable for calculating in real time the data P[x(t), y(t), z(t)]) encoding the absolute instantaneous 3D position of said sensor and the data A[α(t), β(t), θ(t)] encoding the absolute instantaneous 3D angle of inclination of said sensor in a predefined three-dimensional reference point (X, Y, Z).

The predefined three-dimensional reference point (X, Y, Z) constitutes the frame of reference (Rt) of the magnetic field transmitter 20. During a treatment, the body C is placed in the magnetic field emitted by this transmitter 20, and the treated part of the body C is preferably immobile in the reference point (X, Y, Z) for the duration of the treatment.

In a further embodiment, the frame of reference (Rt) can be linked to the part of the human or animal body, for instance by fastening the magnetic field transmitter 20 to the part of the human or animal body to be treated. In this case, once the zone or volume to be treated has been mapped in the frame of reference (Rt), it is not necessary to restart the mapping in the event of the part of the animal or human body moving.

With reference to FIG. 3, the data P[x(t), y(t), z(t)] on the absolute instantaneous 3D position of the sensor 21 and the data A[α(t), β(t), θ(t)] on the absolute instantaneous 3D angle of inclination of the sensor 21 are treated in real time by the second electronic computation means 22b, the parameters of which are entered with the relative position Pc(dx,dy,dz) of the sensor 21 with regard to the outlet 12a of the optical fibre 12. This relative position Pc of the sensor 21 is fixed in time, regardless of the position and angle of inclination of the instrument 1, and is an information, preferably modifiable, that is for instance stored in a memory of the second electronic computation means 22b.

The second electronic computation means 22b are designed so as to calculate in real time the data P′[x′(t), y′(t), z′(t)] encoding the absolute instantaneous 3D position of the outlet 12a of the optical fibre 12 of the instrument 1 in the three-dimensional reference point (X, Y, Z), from said data encoding the absolute instantaneous 3D position P[X(t), Y(t), Z(t)] and the absolute instantaneous 3D angle of inclination A[α(t), β(t), θ(t)] of the sensor 21, and from the relative position Pc(dx,dy,dz) of the sensor 21.

In the particular embodiment of FIGS. 1 and 3, the first electronic computation means 22a are integrated in an external casing distinct from the above-cited device 13 and the second electronic computation means 22b are integrated in said device 13 and communicate locally with the first electronic computation means 22a via a link 22c (FIG. 1), which can be either wired or wireless.

The transmitter 20, the sensor 21 and the first electronic computation means 22a are known means, and can for instance, and in a non-limiting manner of the invention, consist of components of a magnetic localisation device marketed by the company Ascension Technology Corporation under the brand “Flock of Birds®”. The second electronic computation means 22b can consist of any programmable processing unit, implementing for instance a microprocessor or microcontroller suitable for carrying out a computation programme enabling an absolute instantaneous 3D position P′[x′(t), y′(t), z′(t)] to be calculated from the data of an absolute instantaneous 3D position P[x(t), y(t), z(t)] and an absolute instantaneous 3D angle of inclination A[α(t), β(t), θ(t)].

In a further embodiment, the second electronic computation means 22b can be integrated in the same casing as the first electronic computation means 22a. In a further embodiment, the first 22a and second 22b electronic computation means can be created by using the same computation processor.

### Electronic Dosimetry Control Means 3

The electronic means 3 can be implemented in the form of any type of electronic programmable processing unit including in particular a microprocessor or a microcontroller suitable for automatically carrying out a programme stored in a memory and specific to the invention.

The electronic means 3 receive at their inlet at least the data P′[x′(t), y′(t), z′(t)] encoding the absolute instantaneous 3D position of the outlet 12a of the optical fibre 12, and also, in the particular illustrated embodiment, two signals 13c and 13d delivered by the laser source 13b. The signal 13c enables the electronic means 3 to be informed if a laser shot is being fired or not. This signal 13c is for instance an electric signal of the binary type that can assume two levels, high and low, and that is for instance at the high level when a shot is being fired (activated laser source) and at the low level 0 in the contrary case. The signal 13c is a signal encoding the instantaneous power PUI(t) of the laser source 13b.

In the particular embodiment of FIG. 3, and in an optional manner according to the invention, the electronic dosimetry control means 3 also deliver at the outlet two command signals 3a, 3b enabling them to automatically command the laser source 13b (means of delivering treatment doses). The signal 3a is a signal for regulating the power of the laser source 13b. The signal 3b is a signal enabling the inactivation of the laser source 13b to be commanded, in the event of the detection of an overdose.

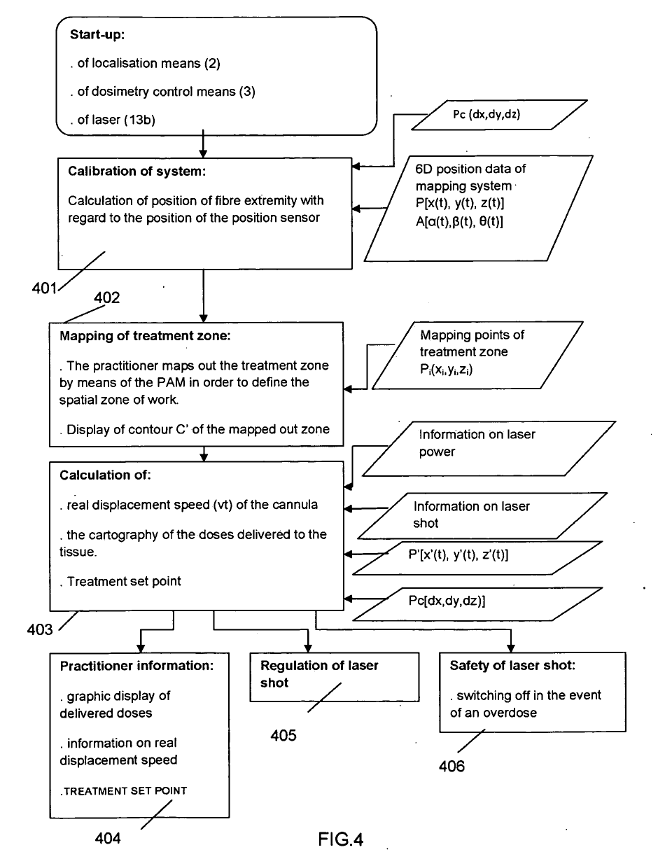

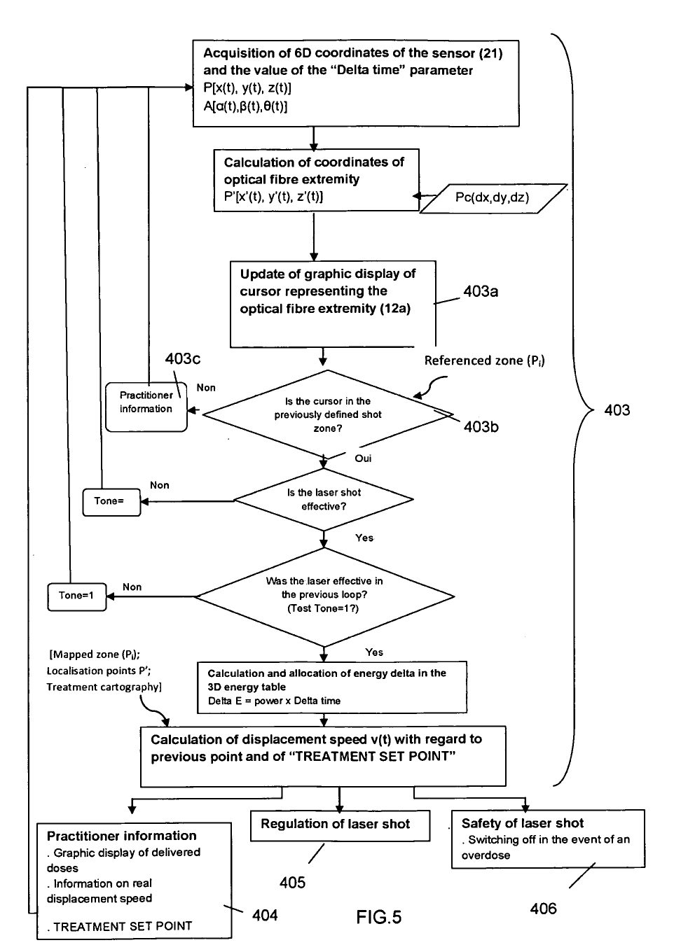

The functionalities of the localisation means 2 and the electronic means 3 will now be described in more detail with the help of the operating algorithms of FIGS. 4 and 5, given by way of non-limiting and non-exhaustive examples of the invention.

### Algorithms of FIGS. 4 and 5—Example of a Treatment Course

Once the localisation means 2, the electronic means 3 and the laser source 13b have started up, the localisation means 2 implement a first calibration stage 401 (FIG. 4). This calibration stage consists in entering the parameters of the relative position Pc(dx,dy,dz) of the sensor 21 with regard to the outlet 12a of the optical fibre 12, such that the second computation means 22b can automatically calculate the instantaneous 3D position P′[x′(t), y′(t), z′(t)] of the outlet 12a of the optical fibre 12, from the data supplied by the first electronic computation means 22a and encoding the absolute instantaneous 3D position P[X(t), Y(t), Z(t)] and the absolute instantaneous 3D angle of inclination A[α(t), β(t), θ(t)] of the sensor 21.

Once the localisation means 2 have been calibrated, the dosimetry control means 3 perform a mapping programme of the treatment zone (FIG. 4—stage 402).

For the implementation of this mapping stage, the practitioner draws on the skin of the patient, for instance by means of a felt-tip pen, the contour C (FIG. 1) of each zone to be treated. With reference to FIG. 1, the zone to be treated consists for instance of the part of the human body situated inside the contour C, and no energy dose is to be delivered into the parts of the human body situated outside this contour C. Then the practitioner sterilises the zone to be treated by applying an antiseptic product onto the skin of the patient.

The mapping programme (FIG. 4—stage 405) consists in making the practitioner enter several mapping points Pᵢ of the contour C, by using the instrument 1 without delivering a treatment dose, in order to localise and define the treatment zone in the frame of reference Rt (reference point (X, Y, Z)). To this effect, the practitioner positions the outlet 12a of the instrument in contact with the skin of the patient at a point of the contour C, and displaces the instrument 1 in contact with the skin of the patient, and by following the contour C. During this displacement, the localisation means 2 supply to the electronic means 3 the absolute instantaneous position in the frame of reference Rt of the outlet 12a of the instrument 1, and the electronic means 3 record under the form of mapping data Pᵢ (xᵢ, yᵢ, zᵢ) several of these absolute instantaneous positions of the outlet 12a of the instrument 1.

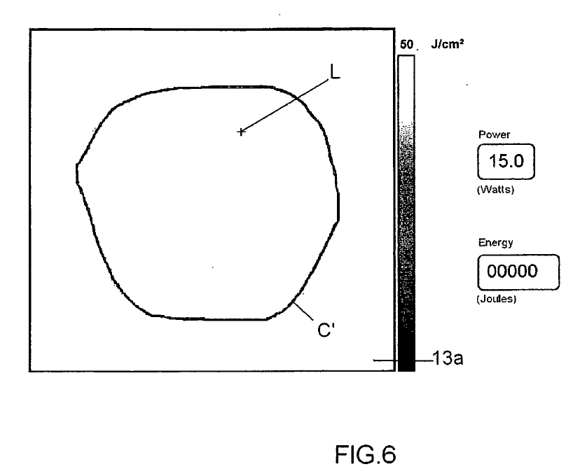

The electronic means 3 use these mapping points Pᵢ (xᵢ, yᵢ, zᵢ) in particular for displaying on the screen 13a the contour C′ of the zone to be treated (FIG. 6) corresponding to the contour C traced on the patient, as and when the practitioner displaces the instrument on the contour C.

This mapping stage of the zone to be treated in the frame of reference Rt is important, since it allows, by means of the mapping points, the frame of reference Rt to be linked to the part of the patient body to be treated.

In a further embodiment, the mapping can be carried out in three dimensions and in an invasive manner by means of the instrument 1, and the control means can be programmed to display on the screen 3 no longer a treatment zone in a plane defined by a contour C′, but a treatment volume in three dimensions. In addition, in a further embodiment, the mapping can be carried out not by means of the treatment instrument 1 but by means for instance of a specific tool dedicated solely to mapping, and enabling mapping points Pᵢ of the zone or volume to be treated to be obtained by the electronic means 3 in the frame of reference Rt. This mapping tool can for instance be a specific pointing device, which differs from the treatment instrument 1 and which is suitable for using to point out mapping points Pᵢ on the zone or volume to be treated of the human or animal body.

Once the calibration 401 and mapping 402 stages have been completed, the treatment device is ready to be used by the practitioner.

By means of the instrument 1, the practitioner carries out the appropriate sub-cutaneous or intra-cutaneous laser treatment in a manner known as such, according to a treatment protocol previously determined by him. During the course of this treatment, the practitioner, using a scalpel, makes one or several incisions in the skin around the zone, and then starts the treatment by introducing, via one of the incisions made, the extremity of the cannula 11/optical fibre 12 unit under the dermal layer, the dermis or into the dermis (depending on the type of treatment) in the treatment zone that has been mapped out. Then the practitioner carries out in a manner known as such the treatment by performing a series of laser shots and by displacing the cannula 11/optical fibre 12 unit in the treatment zone. The practitioner repeats these operations until the entire zone to be treated has been covered with laser shots.

The electronic means 3 are programmed so that, during the treatment, they can automatically detect, in real time, if the instrument 1 is correctly positioned inside the zone to be treated, and so that they can automatically calculate, in real time, three dosimetry control parameters (stage 403): the speed of displacement of the outlet 12a of the optical fibre 12, which is equivalent in this particular example of the instrument 1 to the speed of displacement of the cannula 11; the cartography of the laser energy doses delivered into the tissue, i.e. the laser energy doses delivered at each point of the treatment zone that has been effectively treated; at least one treatment set point (FIGS. 4 and 5: “TREATMENT SET POINT”) for a manual or automatic regulation of the treatment.

FIG. 5 shows a more detailed algorithm for the implementation of stage 403.

### Detection of the Positioning of the Instrument

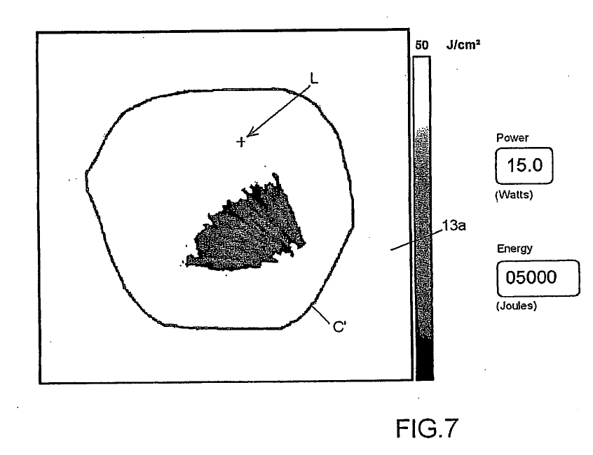

With reference to stage 403a of this FIG. 5, during the course of the treatment, the electronic means 3 use the localisation data of the outlet 12a of the instrument 1 supplied by the localisation means 2 to display in real time on the screen 13a a cursor for the position P′ detected in the frame of reference Rt. In the embodiment of FIGS. 6 and 7, this cursor is represented in the shape of a cross and by reference L.

The localisation means 2 are adapted to localise the outlet 12a of the treatment instrument 1 regardless of the dose treatment delivery by the instrument. The cursor L can thus advantageously be displayed on screen 13a before the practitioner delivers a treatment dose with the instrument. The practitioner can thus visually check in a first step if the cursor L is correctly positioned within the zone that has been mapped out and displayed on screen 13a (contour C′), and can, if needed, correct the position of the instrument outlet 12a. In a second step, once the cursor L is correctly positioned within the zone that has been mapped out and displayed on screen 13a (contour C′), the practitioner operates the laser source 13b, in order to deliver a treatment dose at this position.

With reference to stage 403b of FIG. 5, the electronic means 3 automatically compare the instantaneous position P′ of the outlet 12a of the instrument 1, localised in real time in the frame of reference Rt by localisation means 2, with mapping points Pᵢ of the zone to be treated, and automatically detect if this position P′ is situated inside or outside the treatment zone that has been mapped out.

When the electronic means 3 detect that this position P′ is situated outside the treatment zone that has been mapped out, they inform the practitioner (stage 403c) of such, by for instance triggering an audio or visual alarm, so that the practitioner can, if necessary, rectify the position of the instrument. In a further embodiment, the electronic means 3 can also be designed to automatically command the inactivation of the dose delivery means (laser source 13b) if they detect that this position P′ is situated outside the treatment zone that has been mapped out.

This automatic detection of a correct instrument positioning can be advantageously carried out before starting a laser shot.

### Cartography of Treatment Doses

The calculation of the cartography of the laser energy doses is carried out for each laser shot and in an iterative manner while the laser shot is activated. In this embodiment, this cartography consists in associating, in a 3D table, to each absolute instantaneous 3D position P′[X′(t), Y′(t), Z′(t)] of the outlet 12a of the optical fibre, the dose of electromagnetic radiation delivered to this position, i.e. the sum of the energies delivered to this position (summation of the calculated values of the parameter “Delta E” of FIG. 5 for each position P′[X′(t), Y′(t), Z′(t)]). This cartography of the laser energy doses thus is calculated from the following information:
the data P′[x′(t), y′(t), z′(t)] encoding the instantaneous 3D position of the outlet 12a of the optical fibre 12;
the information that a laser shot is in progress, this information being supplied to the electronic means 3 by the signal 13c;
the power of the laser (parameter “Power” on FIG. 5), this information being supplied to the electronic means 3 by the signal 13d;
the duration of the radiation emission (parameter “Delta time” on FIG. 5).

When necessary, if during the course of the treatment the practitioner carries out several laser shots in the same position, the electronic means 3 calculate the treatment dose (“Delta E”) accumulated for this position.

At each instantaneous position P′ of the treatment instrument detected by the localisation means 2, the calculated treatment dose (“Delta E”) is for instance displayed on the screen 13a (FIG. 7/parameter defined as “Energy (Joules)”).

The electronic means 3 are also programmed to display on the screen 13a, in real time during the course of the treatment, the cartography of the treatment doses delivered at each point, and if necessary accumulated at each point, in relation to the contour C′ of the zone that has been mapped out (FIG. 7). The treatment dose delivered at each point, and if necessary the accumulated treatment dose delivered at each point, is for instance encoded by a colour in function of the level of the dose.

Given that the contour C′ of the zone has been mapped out in the same frame of reference Rt as the localisation points P′ of the doses, the practitioner can advantageously, by just looking at the screen 13a, visually control the localisation of the doses delivered in relation to the zone to be treated, and adapt his movement so as to not leave the zone that is defined on the screen 13a by the contour C′. This display thus enables the movement of the practitioner to be guided, which is particularly useful in the case of an invasive treatment during which the practitioner cannot see the position of the outlet 12a of the dose delivery instrument 1. Also, this display enables the practitioner to check the treatment doses distribution within the zone that has been mapped out, and in particular to assure himself that all the points of the zone inside the contour C′ have been treated with the correct dose, and in the event of the contrary, to redo a shot on the points that have not been treated or the points for which the delivered dose has not been sufficient.

In the illustrated embodiment, the cartography of the laser energy doses delivered into the tissue is a 2D cartography (summation of the energy doses—parameter “Delta E” on FIG. 5) in a predefined plane (for instance, the plane X, Y). In a further embodiment, the cartography of the energy doses can be a 3D cartography or a 1D cartography (summation of energy doses—parameter “Delta E” on FIG. 5) following a predefined axis (for instance axis X).

In a simpler embodiment, the cartography of the doses can be established by recording only the successive positions of the delivered doses (parameter P′[x′(t), y′(t), z′(t)]) without calculating the energy (parameter “Delta E”).

### Real Speed of Displacement of the Instrument

The calculation of the real speed of displacement of the instrument during the course of the treatment is carried out from the 3D instantaneous positions P′[x′(t), y′(t), z′(t)] of the outlet 12a of the optical fibre 12, which are successively calculated at each iteration.

The displacement speed of the outlet 12a of the dose delivery instrument 1 is for instance displayed in real time on a screen 13a of the device 13 (FIG. 4 or 5/stage 404), which enables the practitioner to control in real time during the course of the treatment the displacement speed of the instrument 1 and to manually adapt his movement in such a manner as to respect a minimal displacement speed that he will have predefined and to reduce the risks of overdosing linked to an excessively slow displacement speed. The speed can be displayed in a numeric form or be encoded, for instance by means of a scale of a bar-type graph.

### Treatment Set Point

In the particular embodiment of FIGS. 4 and 5, the electronic control means 3 are programmed to calculate in real time, during the course of a treatment, at least one treatment set point from the mapping data (Pᵢ) of the zone to be treated, the localisation data (P′) of the outlet 12a of the instrument 1 and from a cartography, a so-called “treatment cartography”. This treatment cartography is previously drawn up by the practitioner, by associating with each point of the zone to be treated that has been mapped out (inside of contour C′) a predefined value of a treatment parameter, said treatment parameter being for instance the treatment dose, the speed of displacement of the instrument 1, the power of the dose delivery means 13b. This treatment cartography is recorded in the electronic means 3, prior to the treatment taking place. In its simplest version, this treatment cartography defines a single value of the treatment parameter identical for the entire zone to be treated that has been mapped out. In a more elaborate version, this cartography can define different values of the treatment parameter (dose, speed or power) for different points of the zone to be treated that has been mapped out.

Within the context of the invention and in function of the chosen treatment parameter, the set point that is calculated (“TREATMENT SET POINT”) can be a set point for the treatment dose (energy) to be delivered, a set point for the speed of displacement of the instrument, a set point for the power of the dose delivery means (laser source 13b) or, more generally, a set point relating to any regulating parameter of the dose delivery means.

The treatment set point is calculated by automatically selecting, in the treatment cartography, the value of the treatment parameter associated with the instantaneous position P′ of the treatment instrument 1, which is detected in the frame of reference Rt by the localisation means 2.

### Regulation of Treatment Parameter

In an embodiment of the invention, the treatment set point can advantageously be displayed in real time on the screen 13a in relation to the real value of the treatment parameter measured (dose, speed or power) in such a manner as to allow the practitioner to manually regulate this treatment parameter (for instance modification of the speed of displacement of the instrument, manual regulation of the power of the laser source 13b, manual inactivation of the laser shot in progress) so as to respect the treatment set point displayed on the screen 13a.

In a further embodiment, the electronic means 3 can advantageously be designed to automatically command the treatment dose delivery means 13b, so as to automatically regulate, during the course of the treatment, the functioning of the treatment dose delivery means 13b in order to automatically respect the treatment set point that has been calculated (FIG. 4 or 5/stage 405).

In a further embodiment, the dosimetry control means 3 can advantageously be designed (FIG. 4 or 5/stage 406):
to compare the real measured value of a treatment parameter (dose, speed or power) with the value of the treatment set point calculated from the treatment cartography,
to automatically detect in real time, during the course of the treatment, an overdose, when the difference between the two values exceeds a predefined threshold,
and to automatically command the switching off of the laser source 13b, by means of the above-cited command signal 3b, in the event of a detection of an overdose.

In a further embodiment, the dosimetry control means (3) can advantageously be designed:
to compare the real measured value of a treatment parameter (dose, speed or power) with the value of the treatment set point calculated from the treatment cartography,
to automatically detect in real time, during the course of the treatment, an underdose.

Thanks to these dosimetry control means 3, the treatment can be carried out by the practitioner with a greater degree of safety. In addition, the cartography of the delivered doses in relation to the treatment zone that has been mapped out (mapping point Pᵢ defining the contour C′) can be recorded and possibly used by the practitioner for a follow-up in time of the treatment protocols of a patient.

The invention is not limited to a device for implementing a treatment using intra-cutaneous or sub-cutaneous electromagnetic radiation (visible, infrared, hyperfrequency or radiofrequency region), but can also be implemented to control the energy doses delivered by means of a device including an instrument adapted for the implementation of an endovenous therapy or including an exolaser type instrument adapted for the implementation of a non-invasive external laser treatment applied to the surface of the skin.

The energy source 13b is not necessarily an electromagnetic radiation source, but can for instance be an acoustic energy source, the instrument in this case being designed to deliver the acoustic energy produced by said source.

The invention is also not limited to a laser treatment device, but can more generally be implemented to control any type of treatment doses delivered by means of any known type of medical instrument, it being possible for instance for the doses to be a chemical product or a medicinal product administered to a part of a human or animal body.

There are also treatments of the invasive type such as for instance liposuction, during which an invasive instrument is used to aspirate from a part of the human or animal body a given quantity of cells or tissue, such as for instance a cannula for aspirating fat cells in the particular case of liposuction. As in the above-cited treatments, this type of treatment raises the same problems of dose delivery, and it is important for the effectiveness and harmlessness of the treatment to be able to control not only the cell or tissue quantities withdrawn but also the localisation and distribution, in a frame of reference linked to the human or animal body, of the quantities of cells or tissue that have been withdrawn.

Consequently, in the present document, a “treatment dose” also refers to the quantity of cells or tissue that has been withdrawn at a position of the instrument, in a like manner to the above-cited treatments for delivering treatment doses. The invention can thus also be implemented for invasive type treatments, such as for instance liposuction, during which an invasive instrument is used to aspirate in a part of a human or animal body a given quantity of cells or tissue, such as for instance a cannula for aspirating the fat cells in the particular case of liposuction. In this case, the electronic localisation means enable the instantaneous position of the inlet of the instrument to be automatically localised in a predefined frame of reference Rt, and the calculation of the doses by the electronic dosimetry control means 3 corresponds to the quantity of cells or tissue that has been withdrawn at a position of the instrument.

The invention can also be applied to a treatment device that enables treatment doses to be delivered via an outlet 12a of an instrument at the same that it enables treatment doses to be aspirated via an inlet of an instrument (which can be the same instrument or an instrument different from that delivering the doses). In this case, the localisation means preferably enable the instantaneous position of the outlet of the instrument delivering the doses and the instantaneous position of the inlet of the instrument aspirating the doses to be localised in the frame of reference Rt, and are thus suitable for delivering localisation data P′ encoding the instantaneous position in the frame of reference Rt of the outlet of the instrument delivering the doses, and localisation data P′ encoding the instantaneous position in the frame of reference Rt of the inlet of the instrument aspirating the doses.

In the context of the invention, the localisation of the instantaneous positions P′ in a frame of reference Rt of the outlet 12a of the treatment doses delivery means is not necessarily of the 3D type, but can also be a 2D or 1D localisation. The localisation means 2 that have been described can be replaced by any technical means enabling the instantaneous position of the outlet 12a of the treatment doses delivery means to be localised in a predefined frame of reference (Rt) in the form of localisation data (P′). The invention is thus not limited to the particular structure of the localisation means 2 that have been described only by way of a non-limiting example. For instance, these localisation means 2 can be based on an optical detection, if necessary through the skin, of the treatment dose delivered, and for instance of the luminous spot corresponding to the laser shot.

1. Device for treatment of a part of the human or animal body, said device comprising:
treatment dose delivery means enabling treatment doses to be delivered to a part of the human or animal body,
mapping means enabling a zone or a volume of the human or animal body to be treated to be mapped and spatially defined in a predefined frame of reference (Rt) and in the form of mapping data (Pᵢ),
localisation means enabling the instantaneous position of the treatment dose delivery means outlet to be localised in the form of localisation data (P′) in the said frame of reference (Rt),
electronic control means, which, on the one hand, are suitable for recording said mapping data (Pᵢ) of at least one zone or one volume of the human or animal body to be treated, acquired with the help of said mapping means, and which, on the other hand, enable a treatment to be controlled by using, during the course of the treatment, at least said mapping data (Pᵢ) and said localisation data (P′).

2. Device according to claim 1, in which said localisation means are adapted to localise the instantaneous position of the treatment dose delivery means outlet, regardless the dose treatment delivery.

3. Device according to claim 2, in which said electronic control means are informed by a signal if a dose treatment is being delivered or not by the treatment dose delivery means.

4. Device according to claim 1, in which said mapping means include a pointing device enabling to point out, on or in the human or animal body, points of the zone or volume to be treated that correspond to said mapping data (Pᵢ).

5. Device according to claim 4, in which said treatment dose delivery means comprise a treatment instrument and the said pointing means are constituted by the said instrument.

6. Device according to claim 1 in which the electronic control means are suitable for displaying on a screen, from said mapping data (Pᵢ), the zone or the volume of the human or animal body that has been mapped out, and to display on said screen during the course of the treatment a cursor that represents the instantaneous position of the outlet of the treatment dose delivery means, which is localised by the localisation means.

7. Device according to claim 1, in which the electronic control means are suitable for displaying on a screen, from said mapping data (Pᵢ), the zone or the volume of the human or animal body that has been mapped out, and to display during the course of the treatment, from at least said localisation data (P′), a cartography of the instantaneous positions of the delivered treatment doses.

8. Device according to claim 1, in which the electronic control means are suitable for calculating, during the course of a treatment, for each instantaneous position (P′) localised by the localisation means, the treatment dose delivered, and if necessary accumulated, during the course of the treatment.

9. Device according to claim 8, in which the control means are suitable for displaying on a screen, from said mapping data (Pᵢ), the zone or the volume of the human or animal body that has been mapped out, and for displaying on said screen, during the course of the treatment, a cartography of the treatment doses by associating each calculated treatment dose to its instantaneous position (P′) localised by the localisation means.

10. Device according to claim 1, in which the electronic control means are suitable for calculating, during the course of a treatment, at least one treatment set point from at least said mapping data (Pᵢ) and said localisation data (P′).

11. Device according to claim 10, in which the treatment set point is selected from the following group: treatment dose, displacement speed of the treatment dose delivery means, regulating parameter of the treatment dose delivery means, power of the treatment dose delivery means.

12. Device according to claim 10, in which the electronic control means are suitable for displaying on a screen the treatment set point that is calculated and the real value of the treatment parameter that is measured during the course of the treatment and corresponds to this treatment set point.

13. Device according to claim 10, in which the electronic control means are suitable for automatically commanding the treatment dose delivery means, in function of said treatment set point, and in such a manner as to automatically regulate during the course of treatment the delivered treatment dose.

14. Device according to claim 1, in which the electronic control means are suitable for commanding the treatment dose delivery means, in function of said mapping data (Pᵢ) and said localisation data (P′).

15. Device according to claim 1, in which the electronic control means are suitable for automatically detecting, from localisation data (P′) and mapping data (Pᵢ), whether the instantaneous position (P′) of the outlet of the treatment dose delivery means that is localised by the localisation means is situated outside or inside the zone or volume that has been mapped out.

16. Device according to claim 15, in which the electronic control means are suitable for automatically commanding the inactivation of the dose delivery means, when they detect that the instantaneous position (P′) of the outlet of the treatment dose delivery means that is localised by the localisation means is situated outside the zone or volume that has been mapped out.

17. Device according to claim 1, in which the electronic control means are suitable for detecting, during the course of a treatment, an overdose or an underdose for each instantaneous position (P′) localised by the localisation means, by taking into account at least said mapping data (Pᵢ).

18. Device according to claim 17, in which the electronic control means are suitable for commanding the treatment dose delivery means, so as to automatically inactivate the treatment dose delivery means in the event of a detection of an overdose.

19. Device according to claim 1, in which said treatment dose delivery means comprise a treatment instrument, and the localisation means are suitable for delivering localisation data P′[(x′(t), y′(t), z′(t)] encoding in the frame of reference (Rt) the instantaneous 3D position of the outlet of the instrument via which the treatment doses are delivered.

20. Device according to claim 1, in which said localisation means include at least one sensor, which is fastened to the instrument.

21. Device according to claim 1, in which said treatment dose delivery means comprise a treatment instrument, in which said localisation means include detection means and electronic computation means, in which the detection means include at least one sensor, which is fastened to the instrument at a different position to the outlet position of the treatment instrument, and are suitable for supplying, to the electronic computation means, data P[x(t), y(t), z(t)] encoding the absolute instantaneous 3D position of said sensor, and the data A[α(t), β(t), θ(t)] encoding the absolute instantaneous 3D angle of inclination of said sensor in a predefined three-dimensional reference point (X, Y, Z), and in which the electronic computation means are suitable for calculating the absolute instantaneous 3D position P′[x′(t), y′(t), z′(t)] of the outlet of the instrument from said data of the absolute instantaneous 3D position P[x(t), y(t), z(t)] and of the absolute instantaneous 3D angle of inclination A[α(t), β(t), θ(t)] of the sensor, and from the relative position Pc (dx,dy,dz) of the sensor in relation to the outlet of the instrument.

22. Device according to claim 21, in which the detection means include emission means for emitting a magnetic field, and in which the sensor of the detection means is sensitive to the magnetic field produced by said emission means and is suitable for delivering electrical signals, which are characteristic of its instantaneous position and of its absolute instantaneous angle of inclination in said magnetic field.

23. Device according to claim 1, in which the treatment dose delivery means comprise a treatment instrument that is invasive.

24. Device according to claim 20, in which the instrument includes a so-called invasive part, which comprises the outlet of the instrument and which is adapted for introduction into a part of the human or animal body, and in which said sensor is positioned in another non-invasive part of the instrument.

25. Device according to claim 1, in which the treatment dose delivery means comprise a treatment instrument and a source of electromagnetic radiation, and in which the treatment instrument includes an optical fibre, which is connected to said source of electromagnetic radiation, and which enables the electromagnetic radiation produced by said source to be delivered at its distal emission extremity, said distal extremity of the optical fibre corresponding to the outlet of the instrument.

26. Device according to claim 20, in which the treatment instrument comprises a cannula, which is threaded and immobilised on the optical fibre, and in which the sensor is integral with the cannula/optical fibre unit.

27. Device according to claim 1, in which the treatment dose delivery means comprise a source of acoustic energy and a treatment instrument that is suitable for delivering the acoustic energy produced by said source.

28. Device according to claim 1, in which the treatment dose delivery means are adapted for destroying adipose cells in a part of human or animal body.

29. Device according to claim 28, in which the treatment dose delivery means are adapted for a lipolysis treatment.

30. Device according to claim 28, in which the treatment dose delivery means are adapted for an adipocytolysis treatment.

31. Device according to claim 1, in which the treatment dose delivery means are adapted for a sub-cutaneous or intra-cutaneous treatment.

32. Device according to claim 1, in which the treatment dose delivery means are adapted for an endovenous treatment.

33. Device according to claim 1, in which the treatment dose delivery means are adapted for heating the collagen present in the dermis.

34. Device according to claim 33, in which the treatment dose delivery means are adapted to, carry a cosmetic skin remodelling or skin healing treatment through heating the collagen present in the dermis.

35. Treatment method of a part of a human or animal body by means of treatment dose delivery means comprising an instrument, during which:
mapping data (Pᵢ) is obtained that maps out and define, in a predefined frame of reference (Rt), a zone or a volume of the human or animal body to be treated,
the instantaneous position in said frame of reference (Rt) of the treatment instrument outlet is automatically localised during the course of the treatment in the form of localisation data (P′),
treatment doses are delivered by means of the instrument by displacing the instrument in relation to the part of the human or animal body to be treated,
the treatment is controlled by using at least said mapping data (Pᵢ) and said localisation data (P′).

36. Method according to claim 35, during which the localisation of the instantaneous position of the treatment instrument outlet is carried out regardless the treatment dose delivery.

37. Method according to claim 35, during which the stage of acquiring mapping data (Pᵢ) is carried out by pointing out the points of the zone or the volume of the human or animal body to be treated by means of a pointing device.

38. Method according to claim 37, during which the stage of acquiring mapping data (Pᵢ) is carried out by using, by way of a pointing device, the treatment instrument, without delivering the treatment dose.

39. Method according to claim 35, during which a screen displays, from said mapping data (Pᵢ), the zone or the volume of the human or animal body that has been mapped out, and during which said screen displays, from at said localisation data (P′), a cursor that represent the instantaneous position of the treatment dose delivery means outlet.

40. Method according to claim 35, during which a screen displays, from said mapping data (Pᵢ), the zone or the volume of the human or animal body that has been mapped out, and during which said screen displays, from at least said localisation data (P′), a cartography of the instantaneous positions of the delivered treatment doses during the course of the treatment.

41. Method according to claim 35, during which for each localised instantaneous position (P′), during the course of the treatment, the treatment dose delivered and, if necessary, accumulated during the course of the treatment, is calculated.

42. Method according to claim 41, during which, from said mapping data (Pᵢ), the zone or the volume of the human or animal body that has been mapped out is displayed on a screen, and a cartography of the treatment doses is displayed on said screen, during the course of the treatment, by associating each calculated treatment dose to its instantaneous position (P′).

43. Method according to claim 35, during which at least one treatment set point is calculated from at least said mapping data (Pᵢ) and said localisation data (P′).

44. Method according to claim 43, in which the treatment set point is selected from following group: treatment dose, speed of displacement of the instrument, regulating parameter of the treatment dose delivery means, power of the treatment dose delivery means.

45. Method according to claim 43, during which the treatment set point, which is calculated, and the real value of the treatment parameter, which is measured during the course of the treatment and which corresponds to this treatment set point, are displayed on a screen.

46. Method according to claim 43, during which the treatment dose delivery means are automatically commanded, in function of said treatment set point, and in such a manner as to automatically regulate during the course of the treatment the treatment dose delivered.

47. Method according to claim 35, during which the dose delivery means are automatically commanded, in function of said mapping data (Pᵢ) and said localisation data (P′).

48. Method according to claim 35, during which, from localisation data (P′) and mapping data (Pᵢ), it is automatically detected whether the instantaneous position (P′) of the treatment dose is situated outside or inside the zone or volume that has been mapped out.

49. Method according to claim 48, during which the dose delivery means are automatically inactivated when it is detected that the instantaneous position (P′) of the treatment dose is situated outside the zone or volume that has been mapped out.

50. Method according to claim 35, during which an overdose or an underdose is detected by taking into account at least said mapping data (Pᵢ) and said localisation data (P′).

51. Method according to claim 50, during which the treatment dose delivery means are commanded in such a manner that the treatment dose delivery means are automatically inactivated in the event of a detection of an overdose.

52. Method according to claim 35, during which the instantaneous 3D position P′[(x′(t), y′(t), z′(t)] of the outlet of the instrument via which the treatment doses are delivered is localised in said frame of reference (Rt).

53. Method according to claim 35, in which the localisation in said frame of reference (Rt) of the instantaneous position of the instrument outlet is carried out by using at least one sensor, which is fastened to the instrument.

54. Method according to claim 52, during which, in order to automatically localise in said frame of reference (Rt) the instantaneous 3D position P′[(x′(t), y′(t), z′(t)] of the outlet of the instrument delivering the doses, a magnetic field is generated, this magnetic field is detected by means of a sensor, which is positioned on the instrument at a relative position Pc (dx,dy,dz) known in relation to the outlet of the instrument, and the absolute instantaneous 3D position P[x(t), y(t), z(t)] of said sensor and the absolute 3D instantaneous angle of inclination A[α(t), β(t), θ(t)] of said sensor are measured.

55. Method according to claim 35, during which the part of the human or animal body is locally radiated by electromagnetic radiation, at different positions of the instrument.

56. Method according to claim 35, during which the instrument is at least partially introduced into the human or animal body, the instrument is displaced in the part of the human or animal body to be treated and the part of the human or animal body is locally radiated by electromagnetic radiation, at different positions of the instrument.

57. Method according to claim 35, during which acoustic energy is delivered locally in the part of the human or animal body, at different positions of the instrument.

58. Method according to claim 35, during which the instrument is at least partially introduced into the human or animal body, the instrument is displaced in the part of the human or animal body to be treated, and acoustic energy is delivered locally to the part of the human or animal body, at different points of the instrument.

59. Use of the device according to claim 1 for a sub-cutaneous or intra-cutaneous treatment.

60. Use of the device according to claim 1 for endovenous therapy.

61. Use of the device according to claim 1 for destroying adipose cells in a part of human or animal body.

62. Use of the device according to claim 1 for a lipolysis treatment.

63. Use of the device according to claim 1 for an adipocytolysis treatment.

64. Use of the device according to claim 1 for heating the collagen present in the dermis.

65. Use of the device according to claim 1 for a cosmetic skin remodelling or skin healing treatment through heating the collagen present in the dermis.

66. Use of the method according to claim 35 for a sub-cutaneous or intra-cutaneous treatment.

67. Use of the method according to claim 35 for endovenous therapy.

68. Use of the method according to claim 35 for destroying adipose cells in a part of human or animal body.

69. Use of the method according to claim 35 for a lipolysis treatment.

70. Use of the method according to claim 35 for an adipocytolysis treatment.

71. Use of the method according to claim 35 for heating the collagen present in the dermis.

72. Use of the method according to claim 35 for a cosmetic skin remodelling or skin healing treatment through heating the collagen present in the dermis
